(19) United States
(12) Plant Patent Application Publication
(10) Pub. No.: US 2021/0144896 P1
(43) Pub. Date: May 13, 2021

(54) RASPBERRY PLANT NAMED 'ENDURANCE'

(50) ;

(71) Applicant: PLANT SCIENCES, INC., Watsonville, CA (US)

(72) Inventor: Scott W. ADAMS, Watsonville, CA (US)

(73) Assignee: PLANT SCIENCES, INC., Watsonville, CA (US)

(21) Appl. No.: 17/093,282

(22) Filed: Nov. 9, 2020

Related U.S. Application Data
(60) Provisional application No. 62/934,263, filed on Nov. 12, 2019.

(51) Int. Cl. A01H 6/74

(52) U.S. Cl. PLT/204

(57) ABSTRACT

This invention relates to a new and distinct everbearing variety of raspberry plant named ‘ENDURANCE’. The new variety is primarily adapted to the growing conditions of the central coast of California and is characterized by the following: late-season primocane production with medium to large sized fruit of medium-red coloration. Fruit is of consistent conic shape, releases easily from receptacle, possesses medium gloss and fair flavor. Foliage is flat to slightly convex, medium green; possessing weak rugosity, weak gloss and equal 3-5 foliates. Primocanes have an absent or very weak waxy coat, medium thorn density of small purple spines and have very weak anthocyanin coloration.

### CROSS-REFERENCE TO RELATED APPLICATION

This application claims priority to and the benefit of U.S. Provisional Application Ser. No. 62/934,263 filed Nov. 12, 2019, entitled “Raspberry Plant Named ‘PS-08.056-18’”, the entire disclosure of which is incorporated by reference herein.

Latin name of the genus and species of the plant claimed: Rubus idaeus.

Variety denomination: ‘ENDURANCE’.

### BACKGROUND

The present invention relates to a new and distinct everbearing raspberry variety designated ‘ENDURANCE’. This new variety is a result of a controlled cross made in 2008 in Watsonville, Calif. between raspberry variety ‘GRANDEUR’ (patented, U.S. Plant Pat. No. 20,459) as the female parent and raspberry variety ‘04.3891’ (unpatented) as the male parent in an ongoing breeding program. The variety is botanically known as Rubus idaeus.

The seedling resulting from the aforementioned cross was asexually propagated by dormant canes in Santa Cruz County, Calif. and was subsequently selected by the inventor from a controlled breeding plot in Watsonville, Calif. in 2010. After its selection, the new variety was further asexually propagated by dormant canes, roots and non-dormant root shoot cuttings in Santa Cruz County, Calif., San Joaquin County, Calif. and Siskiyou County, Calif. The new variety was then extensively tested over the next several years in fruiting fields in Santa Cruz County, Calif. This propagation has demonstrated that the combination of traits disclosed herein as characterizing the new variety are fixed and remain true to type through successive generations of asexual reproduction.

### SUMMARY

‘ENDURANCE’ is primarily adapted to the climate and growing conditions of the central coast of California. This region provides the necessary year-round temperatures required for it to produce and maintain a strong vigorous plant and to remain in fruit production from July through December on primocanes and in the ensuing year from May through July on the floricanes. The following traits have been repeatedly observed and are determined to be unique characteristics of ‘ENDURANCE’, which in combination distinguish this raspberry plant as a new and distinct variety:
1. Dark purple spines
2. Late fruit yield
3. Medium red fruit color
4. Absent to very weak primocane glaucosity
5. Fair flavor

The raspberry variety that is believed to be most closely related to the new raspberry variety ‘ENDURANCE’ is the raspberry variety ‘GRANDEUR’ (patented, U.S. Plant Pat. No. 20,459). In comparison to the similar raspberry variety ‘GRANDEUR’, ‘ENDURANCE’ differs by the following combination of characteristics described in Table 1:
Comparison between Endurance and Grandeur:

TABLE 1

| | | ‘GRANDEUR’ |
|---|---|---|
| Characteristic | ‘ENDURANCE’ | (U.S. PP20,459) |
| Primocane waxy coat | Absent to very weak | Absent to very weak |
| (glaucosity) | | |
| Predominate number | Equal 3-5 | Always 3 |
| of leaflets | | |
| Leaf shape (cross section) | Flat to slightly convex | Flat to slightly convex |
| Rugosity | Weak | Strong |
| Color mature fruit | RHS 43A | RHS 42A |
| | Medium red | Medium red |
| Thorn Tip Coloration | N186B | 180C |
| | Greyed-purple | Greyed-red |
| Relative number of | 26.9 | 16.2 |
| pedicel thorns | Many | Medium |

‘ENDURANCE’ differs from its parents, ‘GRANDEUR’ and ‘04.3891’, by the following combination of characteristics described in Table 2:
Comparison between Endurance, Grandeur and 04.3891:

TABLE 2

| | | ‘GRANDEUR’ | |
|---|---|---|---|
| Characteristic | ‘ENDURANCE’ | (U.S. PP20,459) | ‘04.3891’ |
| Productivity | High | High | Medium |
| Glossiness (fruit) | Medium | Medium | High |
| Firmness (fruit) | Medium to high | Very high | Medium to low |
| Adherence of | Very weak | Very weak | Medium |
| receptacle | | | |
| Primocane time of | Late | Medium | Medium |
| fruiting | | | |
| Fruit size | Medium to large | Medium | Large |
| Flavor | Fair | Good | Good |

For identification, a series of molecular markers have been determined for this new variety.

### BRIEF DESCRIPTION OF THE DRAWINGS

The accompanying color photographs illustrate the overall appearance of typical specimens of the new raspberry variety ‘ENDURANCE’ at various stages of development as true as reasonably possible with color reproductions of this type. Color in the photographs may differ slightly from the color value cited in the detailed botanical description which accurately describes the color of ‘ENDURANCE’. The depicted plant and plant parts of the new raspberry variety ‘ENDURANCE’ were taken in Watsonville, Calif. and are approximately 2 to 16 months old:

FIG. 1 shows typical leaf shape, foliate and rugosity characteristics of ‘ENDURANCE’ taken in the month of September 2020;

FIG. 2 shows bud, flower and fruit characteristics of ‘ENDURANCE’ taken in the month of September 2020;

FIG. 3 shows typical gloss, coloration, and drupelet formation of primocane fruit of ‘ENDURANCE’ taken in the month of September 2020;

FIG. 4 shows typical primocane and spine coloration of ‘ENDURANCE’ taken in the month of September 2020;

FIG. 5 shows typical dormant bud characteristics of ‘ENDURANCE’ taken in the month of January 2019.

### DETAILED BOTANICAL DESCRIPTION

‘ENDURANCE’ has not been observed under all possible environmental conditions. The characteristics of the new variety may vary in detail, depending upon variations in environmental factors, including weather (temperature, humidity and light intensity), day length, soil type, and location.

The aforementioned photographs, together with the following description of the new raspberry variety ‘ENDURANCE’, unless otherwise noted, are based upon observations taken during the 2016 - 2017 growing season in Watsonville, Calif. Primocane measurements and ratings were taken from plants of ‘ENDURANCE’ dug from a nursery located in Siskiyou County, Calif. during the middle of October 2015 and planted approximately 3 to 4 weeks later in Watsonville, Calif. The approximate age of the observed primocane plants is 7 to 8 months. Floricane measurements and ratings were taken from the same planting of ‘ENDURANCE’ at an approximate age of 16 to 18 months. Yield observations and fruit quality characteristics are averaged from 3 years of data collected from the 2015 through 2018 production seasons. Flower measurements and characteristics are from secondary flowers unless otherwise noted. Fruit characteristics and measurements are from secondary fruit unless otherwise noted. Foliage characteristics and measurements are from 3-foliate foliage unless otherwise noted.

Color terminology where noted follows the R.H.S. Colour Chart Fifth Edition, Royal Horticultural Society, London, United Kingdom (1966).

The following tables 3-7 describe fruit, plant, foliage, flower and pest/disease characteristics of the new raspberry ‘ENDURANCE’ in comparison to the similar raspberry varieties ‘GRANDEUR’ (patented, U.S. Plant Pat. No. 20,459).
Comparison between Endurance and Grandeur:

TABLE 3
FRUIT CHARACTERISTICS

| | | ‘GRANDEUR’ |
|---|---|---|
| Characteristic | ‘ENDURANCE’ | (U.S. PP20,459) |
| Color mature fruit | RHS 42A | RHS 42A |
| | Medium red | Medium red |
| Color achenes | RHS 159A | RHS 159A |
| | Orange-white group | Orange-white group |
| Fruit length (mm) | 27.71 | 22.91 |
| Fruit width (mm) | 23.20 | 20.12 |
| Length/Width ratio | 1.19 | 1.14 |
| | Longer than broad | Longer than broad |
| Seed weight (mg) | 1.76 | 1.75 |
| Drupelets per berry | 95 | 93 |
| Weight of single drupe | 0.071 | 0.043 |
| (g/drupe) | | |
| Drupe length (mm) | 7.45 | 5.25 |
| Drupe diameter (mm) | 4.53 | 3.86 |
| Relative size of drupes | Large | Medium |
| Fruit size | Medium | Medium |
| Fruit per node | 10 | 15 |
| Predominant shape | Conical | Conical |
| Receptacle length (mm) | 24.74 | 19.15 |
| Receptacle diameter (mm) | 12.02 | 10.32 |
| Receptacle color | RHS 155B | RHS 155B |
| | White group | White group |
| Evenness of color (fruit) | Even | Even |
| Glossiness | Medium | Medium |
| Adherence of receptacle | Very weak | Very weak |
| Firmness of flesh | Firm | Very firm |
| Firmness of skin | Firm | Very firm |
| Soluble Solids (% brix) | 9.8 | 10.5 |
| Flavor | Fair | Good |

TABLE 4
PLANT CHARACTERISTICS

| | | ‘GRANDEUR’ |
|---|---|---|
| Characteristic | ‘ENDURANCE’ | (U.S. PP20,459) |
| General: | | |
| Habit | Semi-erect | Upright |
| Plant height (m) | 1.5 | 1.4 |
| Plant width (cm) | 38 | 45 |
| Time to initiate roots (days) | 12-14 | 12-14 |
| Canes per hill | 7.5 | 6.9 |
| Productivity | High | High |
| Self-fruitfulness | Yes | Yes |
| Type of bearing | Everbearing | Everbearing |
| Primocane: | | |
| Color (true) | RHS 145A | RHS 145B |
| | Yellow-green group | Yellow-green group |
| Length (cm) | 153.5 | 143.6 |
| Basal diameter (mm) | 19.26 | 21.80 |
| Diameter central ⅓ (mm) | 13.76 | 12.55 |
| Lateral length at | 27.7 | 43.6 |
| central ⅓ (cm) | | |
| No. fruiting laterals per cane | 11.8 | 17.4 |
| Total nodes per cane | 39 | 31 |
| Internode length at | 38.93 | 48.84 |
| central ⅓ (mm) | | |
| Anthocyanin coloration | RHS 59B | RHS 59B |
| | Red-purple group | Red-purple group |
| Anthocyanin intensity | Medium | Weak |
| Pubescence | Absent | Absent |
| Vegetative bud | 8.70 | 11.01 |
| length (mm) | | |
| Vegetative bud | 5.15 | 6.11 |
| diameter (mm) | | |
| Vegetative bud shape | Widely ovate | Widely ovate |
| Vegetative bud color | RHS 187A | RHS 187A |
| | Greyed-red group | Greyed-red group |
| Strength of waxy | Absent to very weak | Absent to very weak |
| coat (glaucosity) | | |
| Time of flowering | Medium to late | Medium |
| Time of fruiting | Medium to late | Medium |
| Length of fruiting season | Long | Long |
| % of total yield | 38% | 50% |
| Flowering period | Late June to | Late June to |
| | Late November | Late November |
| Harvest period | Late July to | Late July to |
| | Late December | Late December |
| Primocane fruit weight (g) | 4.7 | 3.8 |
| Primocane yield (g/plant) | 1,936 | 2,662 |
| Young Shoots: | | |
| Number (per meter) | 15-20 | 15-20 |
| | Medium | Medium |
| Anthocyanin presence | Present | Present |
| Anthocyanin coloration | RHS 179A | RHS 179A |
| | Red-purple group | Greyed-red group |
| Anthocyanin intensity | Weak | Medium |
| Thorns: | | |
| Thorn presence | Present | Present |
| Thorn coloration (tip) | RHS 187A | RHS 180C |
| | Greyed-purple group | Greyed-red group |
| Thorn coloration (base) | RHS N186C | RHS 145B |
| | Greyed-purple group | Yellow-green group |
| Thorn length at | 1.39 | 0.76 |
| central ⅓ (mm) | | |
| Thorn base at | 1.32 | 1.53 |
| central ⅓ (mm) | | |
| Thorn density per cm | 5.25 | 3.38 |
| at central ⅓ | Medium | Medium |
| Thorn texture | Rigid | Rigid |
| Attitude of the tip | Horizontal | Horizontal |
| Floricane: | | |
| Color (true) | RHS 165B | RHS 164A |
| | Greyed-orange group | Greyed-orange group |
| Length (cm) | 123.4 | 119.8 |
| Width at central ⅓ (mm) | 12.58 | 12.04 |
| Total nodes per cane | 22 | 18 |
| Internode length at | 44.55 | 49.13 |
| central ⅓ (mm) | | |
| Fruiting lateral attitude | Erect | Erect |
| Time bud burst | Medium | Medium |
| Time of flowering | Medium | Medium |
| Time of fruiting | Medium | Medium |
| Length of fruiting season | Medium to long | Medium to long |
| % of total yield | 62% | 50% |
| Flowering period | Late April to | Late April to |
| | Late June | Late June |
| Harvest period | Late May to | Late May to |
| | Late July | Late July |
| Floricane fruit weight (g) | 4.6 | 3.7 |
| Floricane yield (g/plant) | 3,161 | 2,680 |

TABLE 5
FOLIAGE CHARACTERISTICS

| | | ‘GRANDEUR’ |
|---|---|---|
| | ‘ENDURANCE’ | (U.S. PP20,459) |
| Characteristic | (3 Foliate) | (3 Foliate) |
| General: | | |
| Color of upper surface | RHS N137A | RHS N137A |
| | Green group | Green group |
| Color of lower surface | RHS 190B | RHS 190B |
| | Greyed-green group | Greyed-green group |
| Venation | Pinnate | Pinnate |
| Shape in cross section | Flat to slightly convex | Flat to slightly convex |
| Arrangement | Compound | Compound |
| Relief between veins (rugosity) | Weak | Strong |
| Glossiness | Weak | Weak |
| Number of leaflets/leaf | Equal 3-5 | Always 3 |
| Total leaf length (cm) | 26.1 | 22.5 |
| Total leaf width (cm) | 24.2 | 23.4 |
| Terminal Leaflet: | | |
| Length (mm) | 165.1 | 127.5 |
| Width (mm) | 120.3 | 91.6 |
| Length/width ratio | 1.4 | 1.4 |
| | Longer than broad | Longer than broad |
| Size | Large | Medium to large |
| Shape | Lobed | Cordate |
| Shape of base | Cordate | Cordate |
| Shape of tip | Acuminate | Acuminate |
| Margins | Biserrate | Biserrate |
| Lateral Leaflet: | | |
| Length (mm) | 128.3 | 105.5 |
| Width (mm) | 74.1 | 65.1 |
| Length/width ratio | 1.7 | 1.6 |
| | Much longer than broad | Longer than broad |
| Rachis length (mm) | 41.3 | 36.2 |
| Rachis diameter (mm) | 2.41 | 1.92 |
| Rachis anthocyanin coloration | RHS 184B | RHS 184A |
| of upper surface | | |
| Rachis anthocyanin intensity | Weak | Medium |
| Orientation | Opposite | Opposite |
| Arrangement | Compound | Compound |
| Relative position of lateral leaflets | Free | Touching |
| Shape | Ovate | Ovate |
| Shape of the base | Rounded | Oblique rounded |
| Shape of the tip | Acuminate | Acuminate |
| Margins | Biserrate | Biserrate |
| Petiole: | | |
| Length (mm) | 54.54 | 59.00 |
| Width (mm) | 3.50 | 3.99 |
| Thorn presence | Yes | Yes |
| Thorn orientation | Erect | Erect |
| Anthocyanin coloration of upper surface | RHS 180B | RHS 184C |
| | Greyed-purple group | Greyed-purple group |
| Anthocyanin intensity of upper surface | Absent to very weak | Weak |
| Stipule length (mm) | 12.77 | 10.23 |
| Stipule orientation | Erect | Erect to horizontal |

TABLE 6
FLOWER CHARACTERISTICS

| | | ‘GRANDEUR’ |
|---|---|---|
| Characteristic | ‘ENDURANCE’ | (U.S. PP20,459) |
| Petal color (upper surface) | RHS 155C | RHS 155C |
| | White group | White group |
| Petal color (lower surface) | RHS 155C | RHS 155C |
| | White group | White group |
| Flower diameter (mm) | 27.25 | 22.55 |
| Petal length (mm) | 8.25 | 6.42 |
| Petal width (mm) | 3.42 | 3.11 |
| Petal length/width ratio | 2.41 | 2.06 |
| | Much longer | Much longer |
| | than broad | than broad |
| Petal shape in cross section | Flat | Flat |
| Petal shape (overall) | Ovate | Ovate |
| Petal shape (apex) | Rounded | Rounded |
| Petal shape (base) | Attenuate | Attenuate |
| Petal margin | Entire | Entire |
| Petal texture | Glabrous | Glabrous |
| No. petals/flower | 5.2 | 5.2 |
| No. sepals/flower | 5.2 | 5.2 |
| Sepal length (mm) | 10.80 | 11.20 |
| Sepal width at base (mm) | 4.41 | 6.49 |
| Sepal shape | Deltate | Widely deltate |
| Sepal shape (apex) | Acuminate | Acuminate |
| Sepal coloration | RHS 144A | RHS 144B |
| (upper surface) | | |
| Sepal coloration | RHS 144B | RHS 144C |
| (lower surface) | | |
| Sepal margin | Entire | Entire |
| Sepal texture | Slightly pubescent | Slightly pubescent |
| No. stamen/flower | 116 | 105 |
| Filament length (mm) | 5.19 | 4.45 |
| Filament color | RHS 155C | RHS 155C |
| | White group | White group |
| Pollen quantity | Medium | Medium |
| Pollen color | RHS 196D | RHS 196D |
| Anther length (mm) | 1.16 | 0.79 |
| Anther diameter (mm) | 0.53 | 0.49 |
| Anther coloration | RHS 155C | RHS 155C |
| (pre-dehiscence) | | |
| Anther coloration | RHS 165B | RHS 165B |
| (post-dehiscence) | | |
| Stigma shape | Lobed | Lobed |
| Stigma length (mm) | 0.12 | 0.10 |
| Stigma diameter (mm) | 0.42 | 0.40 |
| Stigma coloration | RHS 145D | RHS 145D |
| Style length (mm) | 3.08 | 2.03 |
| Style diameter (mm) | 0.40 | 0.39 |
| Style coloration | RHS 145D | RHS 145D |
| Ovule length (mm) | 1.56 | 1.51 |
| Ovule diameter (mm) | 1.47 | 1.40 |
| Ovule coloration | RHS 145B | RHS 145B |
| Pedicle length (mm) | 21.55 | 23.86 |
| Pedicle diameter (mm) | 1.51 | 1.47 |
| Pedicle anthocyanin coloration | RHS 181C | RHS 178A |
| Pedicle anthocyanin intensity | Absent to very weak | Medium |
| Relative number of | 26.9 | 16.2 |
| pedicel thorns | Many | Medium |
| Peduncle anthocyanin | Present | Present |
| presence | | |
| Peduncle anthocyanin | RHS 184C | RHS 184A |
| coloration | Greyed-purple group | Greyed-purple group |
| Peduncle anthocyanin | Weak | Medium |
| intensity | | |

TABLE 7
PEST AND DISEASE REACTIONS

| | | ‘GRANDEUR’ |
|---|---|---|
| Characteristic | ‘ENDURANCE’ | (U.S. PP20,459) |
| Spotted wing drosophila | Susceptible | Susceptible |
| (Drosophila suzukii) | | |
| Two spotted spider mite | Susceptible | Susceptible |
| (Tetranychus urticae) | | |
| Grey fruit mold | Susceptible | Susceptible |
| (Botrytis cinerea) | | |
| Powdery mildew | Moderately | Moderately |
| (Podosphaera aphanis var. aphanis) | resistant | susceptible |
| Yellow rust | Moderately | Moderately |
| (Phragmidium rubi-idaei) | resistant | susceptible |

We claim:

1. A new and distinct raspberry plant named ‘ENDURANCE’, as described and illustrated herein.
